(19) United States
(12) Patent Application Publication
(10) Pub. No.: US 2023/0162835 A1
(43) Pub. Date: May 25, 2023

(54) System and Method for Collecting and Analyzing Mental Health Data Using Computer Assisted Qualitative Data Analysis Software

(71) Applicant: Wendy B. Ward, Darien, CT (US)

(72) Inventor: Wendy B. Ward, Darien, CT (US)

(21) Appl. No.: 17/993,065

(22) Filed: Nov. 23, 2022

Related U.S. Application Data
(60) Provisional application No. 63/282,753, filed on Nov. 24, 2021.

(51) Int. Cl. G16H 20/70; G16H 10/20; G16H 50/20

(52) U.S. Cl. CPC G16H 20/70; G16H 10/20; G16H 50/20

(57) ABSTRACT

A gamified computerized mental health assessment and tracking system is disclosed herein. The system comprises an input device with a user interface that provides the patient a mechanism to supply answers and reactions to prompts and queries with a focus on mental disorders. Artificial intelligence performs data analysis on the user's answers and reactions and generates a report transmitted to a third party with information representing the user's mental health condition.

This application claims the benefit of U.S. Provisional Application No. 62/282,753 filed Nov. 24, 2021, the contents of which are incorporated by reference herein in their entirety.

### BACKGROUND

Mental health and suicide statistics among youth in the United States are shockingly high and the trends have been consistently worsening. Suicide is the second leading cause of death among children aged 10 to 24 and the suicide rate among children aged 10 to 14 has tripled from 2007 to 2017.

World Health Organization studies confirm that over 44 million adults are diagnosed with mental health illness and half of these cases present prior to the age of 14. The mental health screening tools and assessments for children were developed in the 1980s and have never been adapted to leverage the knowledge and technologies that are available today. Currently, the mental health screener given to most children is a generic paper-based set of questions that is often filled out by the parent.

### SUMMARY

One embodiment described herein is a computerized system for tracking a patient's mental health condition. This computerized system consists of an apparatus that displays queries and prompts of a computerized mental health assessment in a gamified manner, which include the use of images and avatars to display queries and prompts that differ based on the age of the patient. It includes a user interface that provides the patient with a mechanism to provide answers and reactions to the queries and prompts. An input device is used that enables a computer to collect a first set of data comprising the answers and reactions of the patient. A central processing unit is used to retrieve and execute instructions, the central processing unit also incorporating a computer assisted qualitative data analysis software, the data analysis software including artificial intelligence to analyze answers and reactions of the patient. The system includes a memory including an electronic holding place for the raw data and instructions required for the central processing unit to perform data analysis. An output device is included that enables the computer to transmit to a third party a second set of data, representing the patient's mental health condition, the second set of data being generated by the data analysis software and received from the central processing unit. Guidance is generated for the third party through a first computerized device that utilizes the second set of data. A notification is transmitted to a third party through a second computerized device.

In embodiments, the artificial intelligence in the system analyzes the answers and reactions to the queries and prompts using natural language processing, facial affect, and/or voice intonation. In doing so, the artificial intelligence may be configured to detect and analyze a range of emotions, including but not limited to surprise, anger, happiness, and sadness. The artificial intelligence is also configured to understand multicultural subjects. In embodiments, the artificial intelligence may be located locally or on a cloud server where the data is analyzed. In some instances, the artificial intelligence is operated by a third party who receives 1 to 5 minute or 2 to 3 minute audio and/or video segments that are flagged for analysis. The third party returns the completed output analysis.

Another embodiment disclosed herein is a computer-implemented method of tracking a mental health condition of a patient. The method comprising: displaying gamified queries and prompts pertaining to the patient's mental health status using an assessment platform, the assessment platform comprising a display differing based on the age of the patient; collecting answers and reactions to the queries and prompts of the patient; using artificial intelligence to analyze the patient's answers and reactions to the queries and prompts; calculating a risk-protective score; computer-generated guidance for a third party; and sending a notification to the third party.

In embodiments, the queries and prompts in the method are configured to ask expanded answers. The use of a first computer equipped with a display, user interface, input device, and a second computer equipped with a central processing unit, memory, and data analysis software may be used.

After testing is complete, the summary report in embodiments may include a risk-protective score that reflects a mild, moderate, or severe mental health condition. In embodiments, a notification of testing completion can be sent by an electronic communication, including but not limited to email, traditional and VoIP phone, fax, Short Message Service chat, Internet Relay chat, video chat, and other computerized means of communication. The notification can be received by a third party, which can include a parent, academic institution, hospital, clinic, primary care physician, pediatrician, insurance company, social worker, psychiatrist or other relevant professional. In embodiments, the notification lacks substantive information on the patient's mental health condition so the third party must log into the computer system to access substantive information on the patient's mental health. In other embodiments, substantive information on the patient's mental health can be accessed within the notification received by the third party. In some embodiments, the method of use involves a first computer equipped with a display, user interface, input device, and a second computer equipped with a central processing unit, memory, and data analysis software. The method is practiced at least 50 times to build a centralized database with patient health information for community use.

### BRIEF DESCRIPTION OF THE DRAWINGS

FIG. 1 is a depiction of one embodiment of a system described herein

FIG. 2A shows a second embodiment of a system described herein.

FIG. 2B shows a third embodiment of a system described herein.

FIG. 3 depicts the method steps of one embodiment described herein.

FIG. 4 is a depiction of one embodiment of the output described in the system described herein.

FIG. 5 is a depiction of another embodiment of the output described in the system described herein.

FIG. 6 is a depiction of another embodiment of the output described in the system described herein.

FIG. 7 shows one embodiment of the avatar of the system described herein.

FIGS. 8-10 are a depiction of another embodiment of the output described in the system described herein.

### DETAILED DESCRIPTION

### The Value Proposition:

This assessment revolutionizes mental health screening for children and adults, thereby improving the health and well-being of generations to come. The screener is a gamified tool that engages children and adults in a series of queries that are age-appropriate for individuals aged 4 to 99 years and provides a thorough assessment to doctors, parents, and families. In some embodiments, the screening tool is designed for children and young adults aged 4 to 24 years old. In further embodiments, the screening tool is geared for adults aged 25 to 59 years old. In other embodiments, the screening tool is optimized for elderly adults aged 60 to 99 years old.

The screener incorporates all the current best-in-class strategies to engage children and adults and provide significant innovation around the assessment algorithms which result in a baseline assessment for doctors and families. The screener connects with a robust back-end psychometric algorithm and provides personal data and relevant information to parents and families as well as a recommendation for next steps. The protocols that follow from the assessment are every bit as important as the assessment itself. Further, the screening results provide the baseline for annual screening updates, resulting in a personalized longitudinal data set that is consistent with how doctors treat patients in every other health issue. This 21st century tool further fosters the education of parents and families about the various facets of mental health, encouraging them to seek help early and without judgment.

### Definitions

As used herein, the term “score” means a number, letter or other image or indicia on a scale representing factors indicative of a patient's mental health condition. A “risk-protective score” is a measurement of the amount of environmental risk factors, personal risk factors and social determinants of health that inform an individual's overall future health conditions most specifically as it relates to childhood development and co-occurring mental illness. Some elements considered include adverse childhood experiences/trauma exposure (ACE), social determinants, relationships, functioning (school, home, peers), parenting, parental mental health, family disruptions, environmental risks (fear of discrimination), as well as a focus on Positive Childhood Experiences (PCE), which can include strengths, gifts, and talents.

The term “computer” means an electronic device equipped with a display, user interface, input device, central processing unit, memory, data analysis software, and output device. Non-limiting examples of computers include desktops, laptops, smartphones, and tablets.

The term “mental health data” refers to both the individual data inputs that are collected throughout the assessment and the data outputs that are calculated based on the data inputs.

The terms “assessment,” “screener,” “tool,” “model,” “application,” and “web-based platform” refer to the invention that is the subject of this patent application.

### DRAWINGS

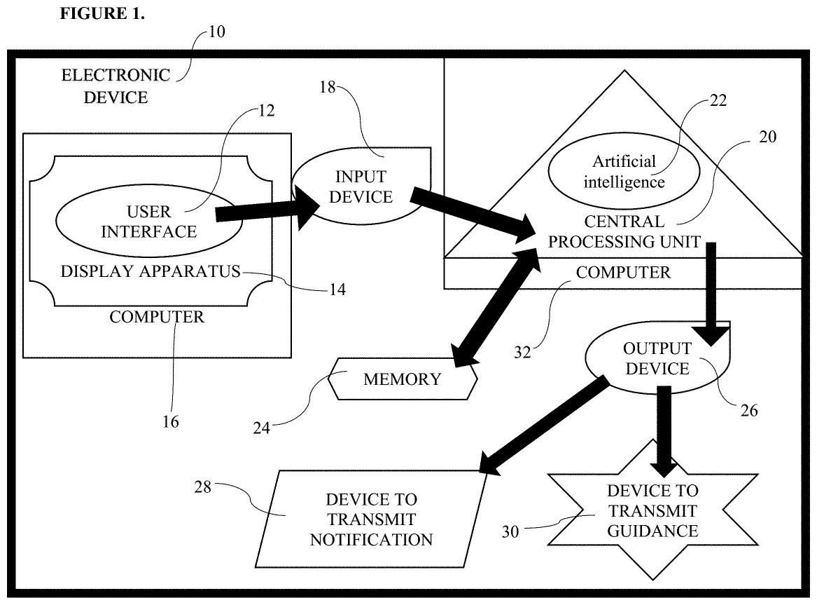

Referring to the drawings, FIG. 1 illustrates an electronic device 10 with the following parts: a display apparatus 14 and a user interface 12 within a computer 16, allowing the patient to interact with the software running the assessment; an input device 18 which allows the electronic device to take in data and send it to the central processing unit 20 within in a computer 32. Within the central processing unit 20 there is artificial intelligence 22 which analyzes data. The memory 24 holds the data and instructions so the central processing unit 20 can perform the data analysis; the output device 26 takes the analyzed data from the central processing unit 20 and allows a device to transmit notification 28 or a device to transmit guidance 30 to send the data over a network to a third party.

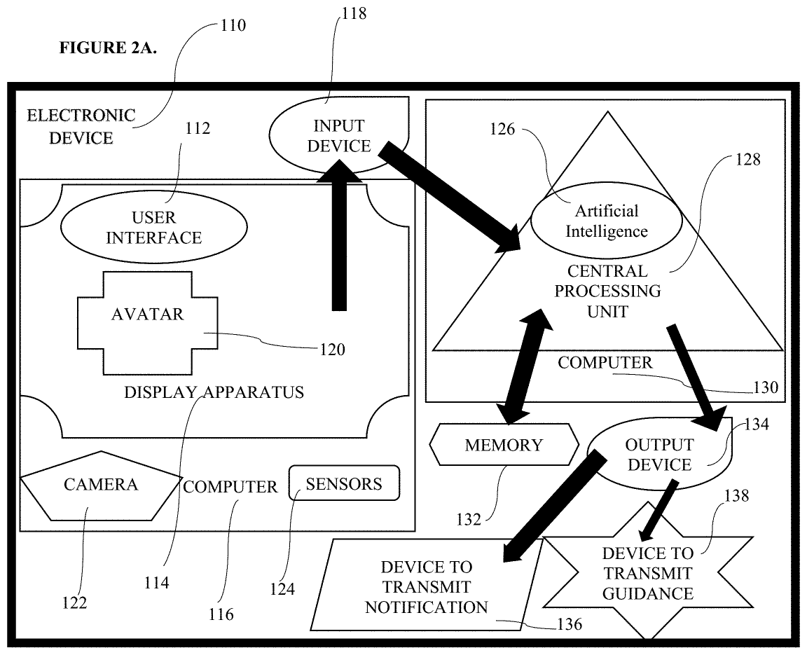

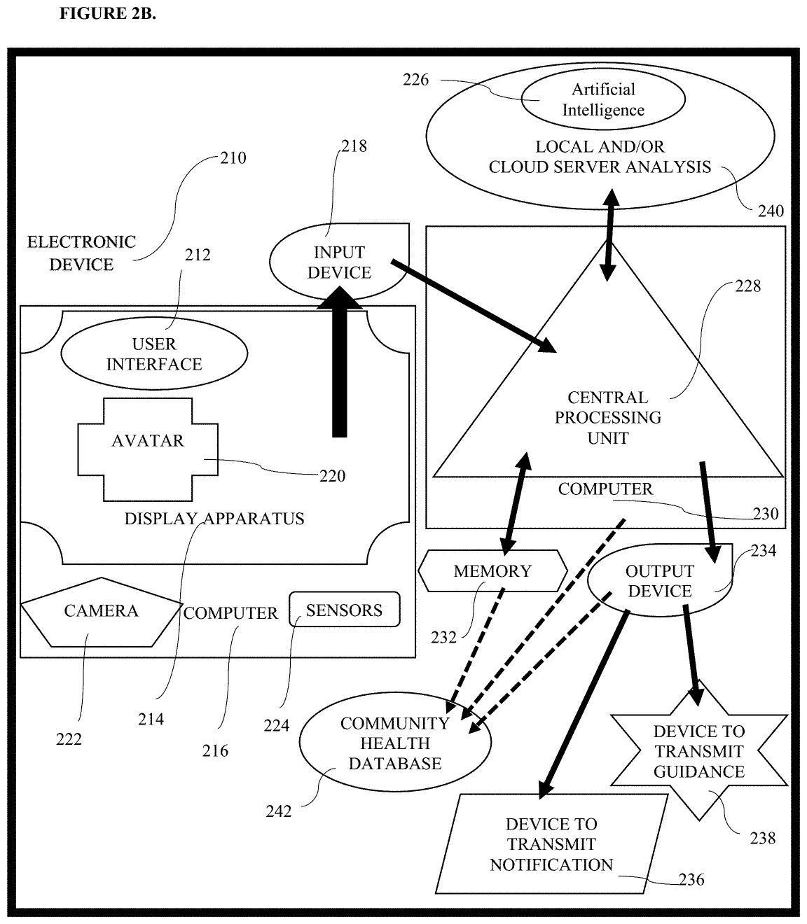

FIG. 2A illustrates an electronic device 110 in a second embodiment with several additional components. The electronic device includes a computer 116 with a display apparatus 114 configured to display an avatar 120. The device 110 also includes a user interface 112 allowing the patient to interact with the software running the assessment and controlling the avatar 120. A camera 122 and sensors 124 associated with the computer 116 allow the assessment to collect audio and visual data. An input device 118 allows the electronic device 110 to take in data and send it to the central processing unit 128 within in a computer 130. The central processing unit 128 employs artificial intelligence 126 which analyzes data in 20 minutes or less, or about 15 seconds to about 15 minutes, or about 1 minute to about 12 minutes. A memory 132 holds the data and instructions so the central processing unit 128 can perform the data analysis. The output device 134 takes the analyzed data from the central processing unit 128 and allows a device to transmit notification 136 or a device to transmit guidance 138 to send the data over a network to a third party.

FIG. 2B illustrates an electronic device 210 in a third embodiment with several additional components. The electronic device includes a computer 216 with a display apparatus 214 configured to display an avatar 220. The device 110 also includes a user interface 212 allowing the patient to interact with the software running the assessment and controlling the avatar 220. A camera 222 and sensors 224 associated with the computer 216 allow the assessment to collect audio and visual data. An input device 218 allows the electronic device 210 to take in data and send it to the central processing unit 228 within in a computer 230. The central processing unit 228 employs artificial intelligence 226 which analyzes data. The artificial intelligence 226 is located on at least one local server and/or cloud server 240 and analyzes data in 20 minutes or less, or about 15 seconds to about 15 minutes, or about 1 minute to about 12 minutes before transmitting the processed data back to central processing unit 228. A memory 232 holds the data and instructions so the central processing unit 228 can perform the data analysis. The output device 234 takes the analyzed data from the central processing unit 228 and allows a device to transmit notification 136 or a device to transmit guidance 238 to send the data over a network to a third party. In one embodiment, the output device 234 transmits the analyzed data to a centralized community health database 242. In another embodiment, memory 232 transmits the analyzed data to a centralized community health database 242. In yet another embodiment, computer 230 transmits the analyzed data to a centralized community health database 242.

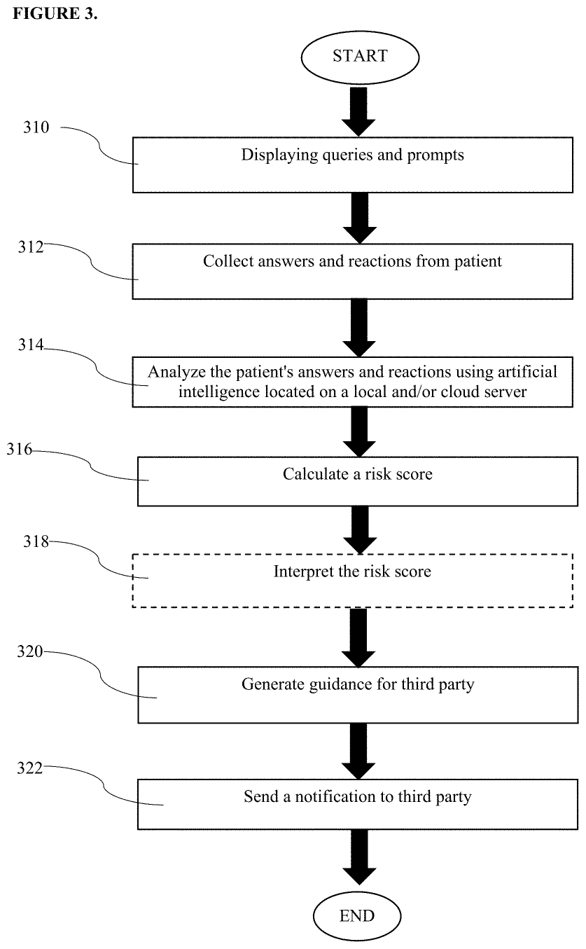

FIG. 3 illustrates the method steps of one embodiment described herein. In the first step 310, the system displays queries and prompts that the patient will be asked to answer and respond to. Next, the system collects answers and reactions from the patient at 312. Subsequently, the patient's answers and reactions to the queries and prompts are analyzed at 314. An overall risk score, made up of multiple scores indicative of the patient's mental health status is calculated at 216 and interpreted 318. Guidance is then generated for a third party 320 and a notification of completion of the assessment is sent to a third party 322.

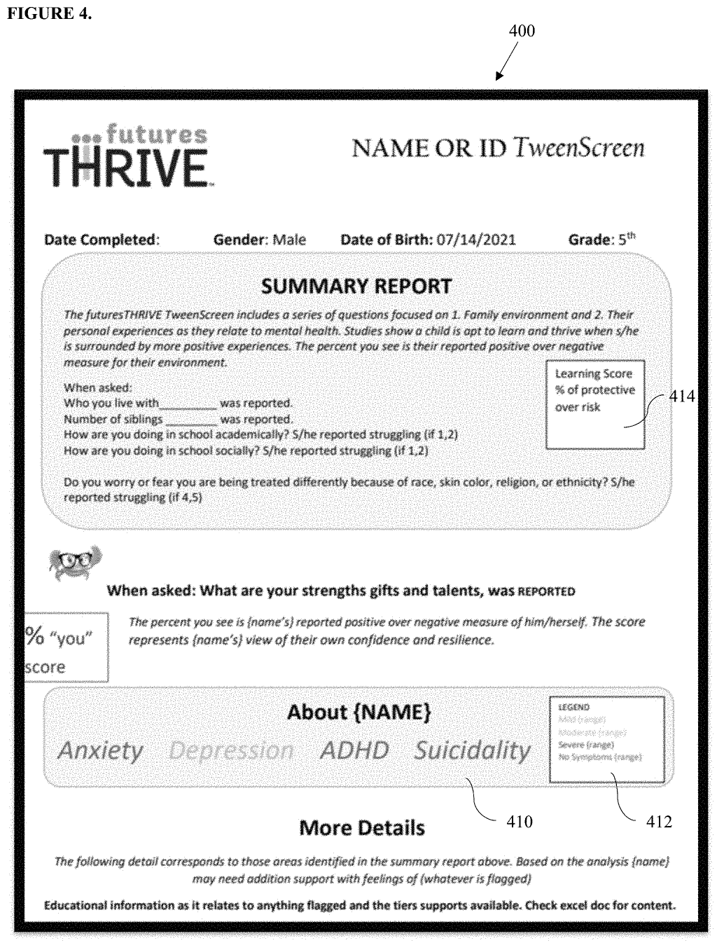

FIG. 4 depicts one embodiment of the assessment output being a report 400 generated for a school. This report highlights the various mental health conditions 410 the patient could be experiencing. The mental health conditions 410 are color coded according to legend 412, which correspond to a range of no symptoms to severe symptoms. Additionally, a learning score 414 represents an individual's preparedness for learning. In one embodiment the learning score is a positive over negative measure for their environment but there are alternative ways to calculate this.

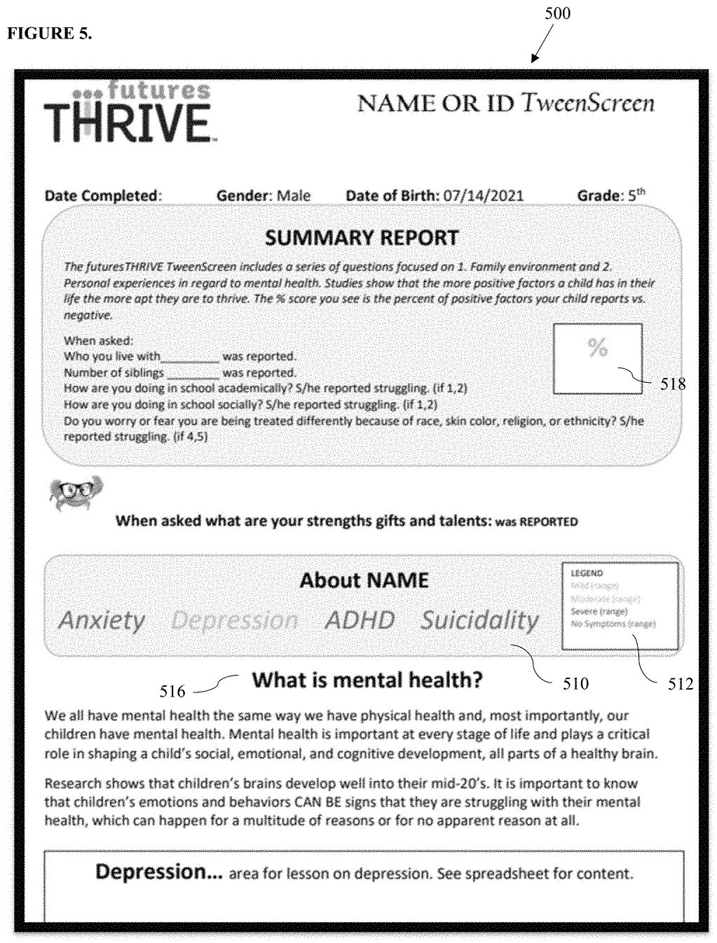

FIG. 5 depicts another embodiment of the assessment output being a report 500 generated for a parent. This report highlights the various mental health conditions 510 the patient could be experiencing. The mental health conditions 510 are color coded according to legend 512, which correspond to a range of no symptoms to severe symptoms. Additionally, this report includes a comprehensive description 516 explaining mental health. In one embodiment, the score 518 reported represents the percent of positive factors an individual reports versus negative but there are alternative ways to calculate this.

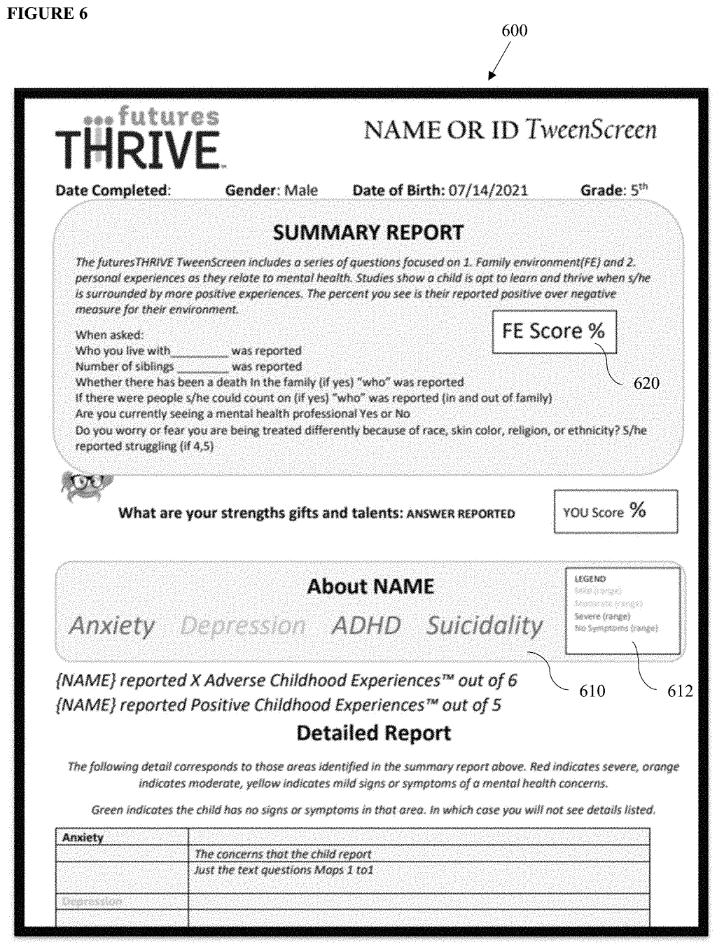

FIG. 6 depicts another embodiment of the assessment output being a report 600 generated for a doctor. This report highlights the various mental health conditions 610 the patient could be experiencing. The mental health conditions 610 are color coded according to legend 612, which correspond to a range of no symptoms to severe symptoms. Additionally, a family environment score 620 represents relational and social determinants of health. In one embodiment the family environmental score 620 is a positive over negative measure for their environment but there are alternative ways to calculate this.

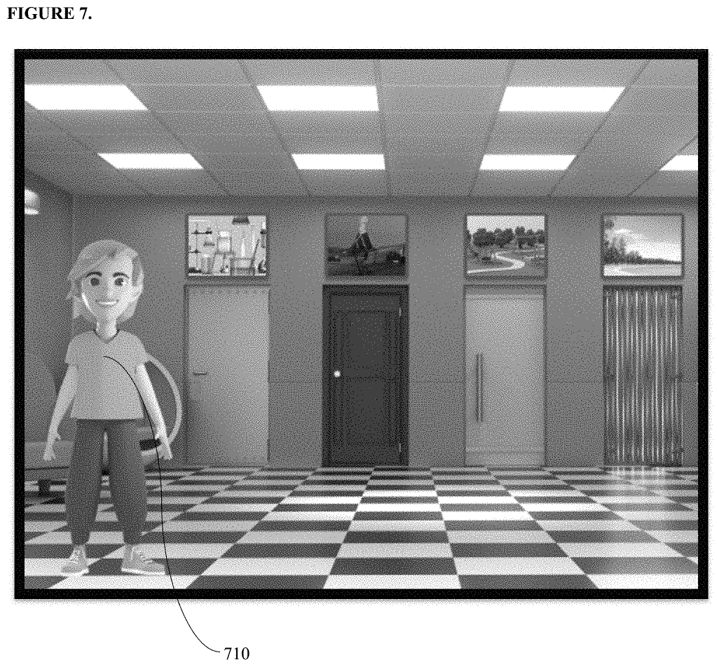

FIG. 7 depicts one embodiment of an avatar 710 as it will be displayed for the assessment.

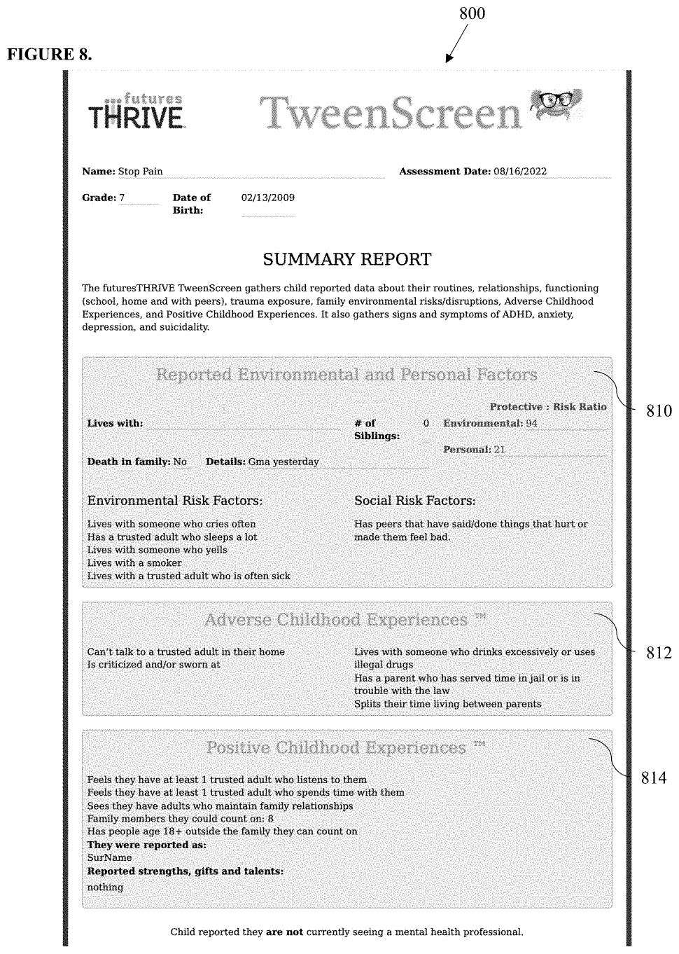

FIG. 8 depicts another embodiment of the assessment output being a summary report 800. The report summarizes the user's screening test responses by displaying specific environmental and personal factors 810, Adverse Childhood Experiences (ACE) 812, and Positive Childhood Experiences (PCE) 814.

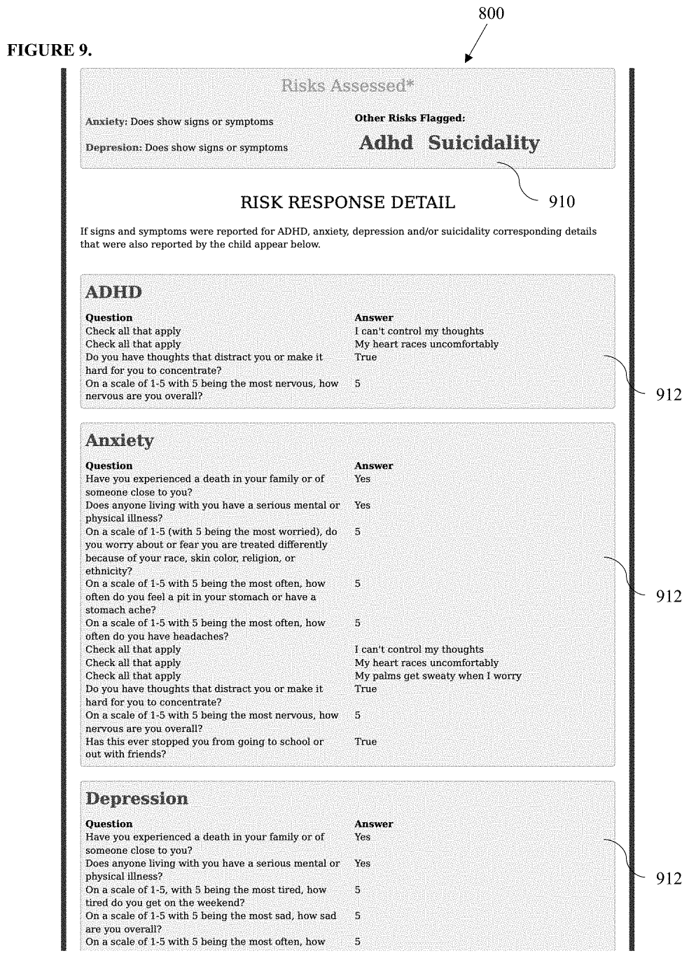

FIG. 9 depicts a continuation of summary report 800 by reporting the types of risks 910 that were assessed to identify the testing basis for the parent or provider. The user's responses to individual risks 910 are further categorized individually with specificity 912.

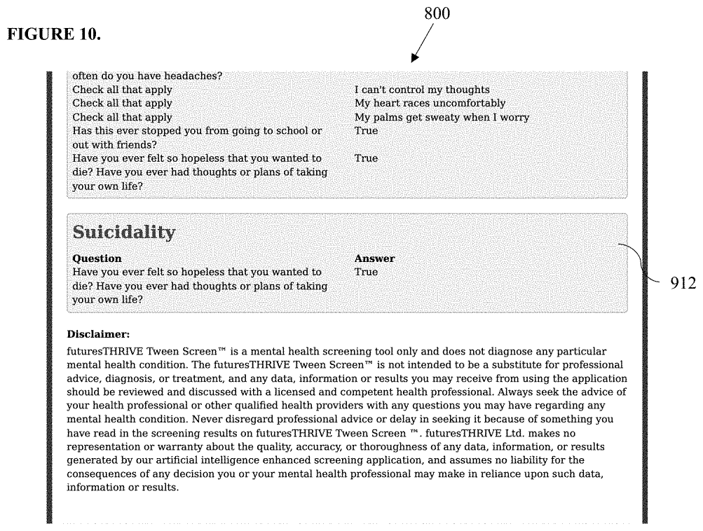

FIG. 10 depicts a further continuation of summary report 800 and displays the user's responses to individual risks 910 are further categorized with specificity 912.

### The Innovation:

This assessment provides a breakthrough approach that engages children and adults using a game-based technology on an iPad, other tablet, or computerized device. It leverages the best of what is available in today's gaming world to engage children and adults to complete these question sets. The questions themselves are updated regularly to include the best data science on mental health. Using artificial intelligence, this assessment adapts to become more predictive as the data set grows.

The innovations include:
using artificial intelligence to collect data while reducing age, gender, and possible cultural biases;
combining questions into one assessment tool to test for one, two, three, or more of the following mental health conditions, including but not limited to anxiety, depression, attention deficit hyperactivity disorder (ADHD), relationships, suicidality, family disruptions, functioning in family, school and peers, Adverse Childhood Experiences (ACE), Positive Childhood Experiences (PCE), and positive findings;
creating the nation's largest mental health data set;
providing quantified mental health data of the community as one entity in a database; and
providing a first-in-class set of automated protocols and recommendations for doctors and parents in real-time.
   As a result, this assessment is the first-ever individualized assessment designed for youth and adults that can be easily launched to have an impact in communities.

### The Commercial Opportunity:

World Health Organization studies state that over 44 million American adults are diagnosed with mental health illness; half of whom present prior to the age of 14. With one in five children currently having a diagnosable mental health condition, the impact potential of a universal screener is extraordinary. It is easy to think that so much work is already being done combat the effects of mental health and substance abuse. In fact, the Substance Abuse and Mental Health Services Administration (SAMHSA) states that mental health and Substance Abuse Disorder treatment spending from all public and private sources is expected to total $280.5 billion in 2020, an increase from $171.7 billion in 2009. These amounts include the effects of the Affordable Care Act.

Yet, suicide is still the second leading cause of death among youth aged 10 to 24. Mental Health America states that 46 percent of Americans will meet the criteria for a diagnosable mental health condition at some point, and 75 percent will develop a condition by the age of 24. Current efforts to combat today's mental health epidemic continue to fall short as evidenced by the CDC's latest figures that show rates of youth suicide and depression on the rise. In fact, the suicide rate among children aged 10 through 14 has nearly tripled from 2007 to 2017, while the suicide rate among older teenagers has increased by 76 percent between 2007 and 2017.

In a preliminary needs assessment done by the American Academy of Pediatrics (AAP), few providers regularly conducted mental health screening, an experience documented across many states. In December 2017, AAP conducted a study using ten practices (107 providers) in which they looked to improve annual mental health screening rates by reducing the perceived burden of current implementation and assessments. The AAP found that when the new program to improve mental health integration was implemented, screening rates increased from a baseline of 1% to 74% by the end of the project.

This AAP study confirms that a learning collaborative model similar to the one here can improve mental health screening practices in pediatric primary care. It is important to note that the AAP study was testing only the efficacy of the learning collaborative model and did not intend to introduce a new product into the market; in fact, the tool used was a standard paper and pencil set of questions. The study highlights the importance of easy-to-use tools and programs to increase the likelihood of early identification. This product is designed explicitly to be used independently by the child or adult, inherently reducing the burden to the practice and provider.

Middle aged adults and the elderly also suffer from mental health disorders including depression, persisting grief reactions, anxiety, and post-traumatic stress disorder. Further increasing the complexity of diagnosing mental health disorders in this population are additional contributing factors such as varying combinations of medical, neurological, psychological symptoms, as well as age-related variability during clinical evaluations. It is also known that many older adults who may be experiencing mental health disorders might not seek care from mental health specialists. Instead, they may seek treatment from a general practitioner which could put the patient at risk of being under-diagnosed and undertreated. Such outcomes can be both financially and socially costly as older adults with depression, anxiety, and other mental disorders have a higher likelihood of being disabled by medical illness, rely more heavily on health care services, and have higher rates of mortality.

Data shows there is a large delay in time between onset of symptoms and proper treatment leaving families stranded with expensive and lengthy challenges caring for affected children or adults. Untreated mental illness is also now the leading cause of disability and contributes to rising costs shouldered by society at an estimated $100 billion annually. Without early monitoring and diagnosis, mental illness can result in developmental delays in social, emotional, and educational milestones that negatively impact the child and family combined with considerable expenses in medical costs. With older adults, early and accurate identification of mental health disorders would help families and or social services provide the necessary support services.

Clearly, it is evident that a primary pain point of mental health treatment is lack of early identification. The compounding challenge in early assessment is both the lack of training and availability of scalable assessment tools even though decades of research show that early intervention is by far the most effective means of preventative care. This assessment offers a solution to this problem.

Addressable Market for Innovation; Economic and Market Drivers in this Industry:

Doctors, parents, and schools evaluate behavior and screen for mental health beginning in adolescence, if they screen at all. Therefore, the initial market is primary care at the pediatric level.

The National Survey of Children's Health showed that more than 90% of children nationwide had visited a primary care provider at least once in the previous year. Additionally, in a social media survey of 100 respondents we conducted, over 88% of respondents, identifying as parents, would choose a pediatric office with mental health screening capabilities over others. Further, the American Academy of Pediatrics is calling for more mental health screening tools in the market.

Starting at age 4, this mental health screener is built with artificial intelligence analytics for natural language, facial affect, and voice intonation. The first to market metrics allows pediatric offices and families to flag and address concerns early without stigma or judgment before onset. Using this model, the trusted pediatrician and an in-office mental health practitioner provide early education and information before issues arise giving families individual baselines for their children. In the event of a rising mental health concern, longitudinal data is available to compare and treat more accurately. This shift in thinking provides a true continuum of care for families in need, all while educating and supporting a new belief system surrounding mental health. Mental health is no longer something considered separate and distinct from physical health: it is an integral part of an individual's overall well-being.

By integrating a high-tech mental health screener, giving actionable interpretations of the data to doctors, and using groundbreaking protocols, this assessment is expected to change the trajectory of suicide rates.

Using an optional annual subscription model to sell directly to doctors, schools, and practitioners, this assessment offers a comprehensive program that integrates the practice of mental health care and pediatric care. By combining the power of artificial intelligence with human wisdom and heart, this tool will make pediatric mental health screening routine for every child while educating America that a cared-for mind is vital to a healthy life. This assessment will help reveal that the mind is at the heart of health. By normalizing mental health care and removing its stigma, the three stakeholders of healthcare (doctors, insurers, and families) will have in hand information and technology to flag and inform treatment of mental health concerns earlier and regularly. In some embodiments, the revenue model consists of an upfront implementation fee for practices and a recurring software fee for users.

### Competitive Landscape and Change:

In speaking with doctors and practitioners there is an expressed desire to have more dynamic mental health assessments that are easier to use than today's paper-based tools. Covid-19 has escalated mental health concerns and necessitated a greater level of comfort with telehealth and other health technology tools. This landscape, combined with the advances in artificial intelligence primes the market for more 21st-century mental health tools.

### Intellectual Merit

First-of-its-kind Engine: In some embodiments, communication occurs through both verbal and non-verbal means. In fact, experts estimate that 85% of human communication is non-verbal. To be able to collect relevant data in a scalable and accessible manner, this web-based platform can be configured to incorporate the following innovations:
A groundbreaking game to collect data points from children and young adults never previously collected. The gamification engages children and young adults in an experience they are comfortable with to survey signs and symptoms of mental health concerns.
Advance use of facial affect and voice intonation to create a personalized recommendation to inform trusted adults.
Machine learning to create new keyword data lists that include words and phrases from a multitude of cultural backgrounds and research theories like the Adverse Childhood Experience (ACE Study) and Positive Childhood Experiences (PCE) work.

Gamification to create a large mental health data set: Best healthcare practices are able to identify early signs of risk, yet mental health measurements consistently focus on self or parent-reported signs and symptoms. This leads to a report primarily focused on behavior, which in turn directs behavior-based solutions. Medical Advisory Board members have collaborated with the software development team to design and provide open-ended questions that are aimed at garnering data points for anxiety, depression, risk factors, and protective factors. Expanding on a widely used therapeutic practice of “play therapy” where children are relaxed and feel freer to express themselves, games offer a real glimpse into the child's state of mind. This therapy style is woven into questions that direct the child in play. Video games are increasingly used as a method of therapy but largely untested in mental health assessments. Questions in some instances may ask for expanded answers: “What makes you sad?” versus today's “Are you sad, Always, Sometimes, Never.”

Facial recognition and voice intonation: This assessment has a machine learning tool to recognize cultural differences that could not be mitigated without the use of technology/artificial intelligence. Today's facial recognition can identify a person and can even identify if they are wearing a mask. However, algorithms are trained primarily using white adult subjects. This team has built the initial data set using multicultural data. To do this, there is a multicultural team and beta testing in multicultural settings among socioeconomically diverse populations. This minimizes bias as the machine learning algorithm is trained.

By using emotion recognition and voice biomarkers the machine can learn to identify a connection to natural language. Using the question above: If the child reports mom and dad getting a divorce makes her sad, the machine can identify if she is also crying or her voice is shaking. To do this, the medical advisory team identifies a set of emotional responses of importance to mental health and the developers build a coded identification system based on facial measurements to identify the desired emotion including but not limited to surprise, anger, happiness, and sadness. Voice markers are flagged and cited as further data for assessment context. In some cases, voice recognition is used to transcribe the person's speech to text for later evaluation and reference.

Machine Learning to generate proprietary eKeys list: Prior known assessment tools ask for limited data points. By collecting natural language data, children and young adults have the opportunity to disclose anything they are experiencing or processing. The use of speech to text keyword flagging helps to identify risk factors that don't currently exist on paper/pencil screenings. This machine learning algorithm is built using a dataset of science-backed words and phrases that research shows are problematic. A diverse team of medical advisors inform the keyword list and draw from suicide research, mental illness research, ACE study research among others. An extensive sample of words and phrases are used to train algorithms for Beta testing. For example: “I cut my wrists when I am scared.” Cut my wrists would be flagged. These algorithms learn as they collect linguistic usage, word patterns and pronunciation, training the machine learning algorithm to become more comprehensive over time. Gender and race classifications inform diversity. This type of personalized assessment can significantly shorten the 9-year average from onset to a proper diagnosis.

The embodiments disclosed herein provide a gamified platform can be used to collect, track, and identify signs and symptoms allowing doctors and families to treat early. The principles behind this are not simple. While engaged in the game, youth interact with a web-based tool using natural language and the computer logs natural language, facial expressions, voice shifts, and measure sentiment.

This simplified, engaging platform is created in collaboration with child psychology experts and gaming exerts. This platform gathers the verbal and non-verbal data points through a themed interview using wording and question sequence which minimizes the textual complexity for youth. The design of the platform is key to the overall success of the feasibility study.

Question sequences are developed to become increasingly more personal as the child gets more comfortable during the session. Professionals watch video interviews and track emotions and voice shifts compared to that of the machine response.

The embodiments disclosed herein provide a machine that can adequately identify emotion and voice shifts. This is because of the beta testing taking place in a controlled environment with a third-party observer. Data points are compared and where there are discrepancies with the computer, the notes from the observer inform the programming team.

The eKeys selected yield superior scoring and reduce bias It is important to create an assessment that can reduce bias and not create more stigma in regard to mental health treatment and services. Multiple factors of measurement are applied, including frequency of terms used by the user, escalation of words and phrases, and overall sentiment of the user. A multicultural, science-backed, and research driven keyword flagging list is used which accounts for high-level risk, like suicidal thoughts, variables in word patters and pronunciation.

### Technical Discussion

Paper and pencil screeners collect limited information and require human time and cost for interpretation. They are categorized and well validated, hence their use has been beneficial when screening for specific disorders. The breakthrough assessment described herein creates a new screener providing an unprecedented measurement of mental health that provides longitudinal data, reduces bias, is broadly accessible, and encompasses several common mental health concerns in one, including the ability to flag environmental issues often completely absent from current assessment methods.

As science shows, 55% of communication is body language, 38% is the tone of voice, and 7% is the actual words spoken. “When there are inconsistencies between attitudes communicated verbally and posturally, the postural component should dominate in determining the total attitude that is inferred” (Mehrabian & Wiener, 1967 and Mehrabian & Ferris, 1967 Nonverbal Communication). In fact, the accuracy of a treatment path can be increased by applying the 3 C's of Nonverbal Communication: context, clusters, and congruence. Context includes what environment the situation is taking place in. Here, youth are taking a mental health screener in a private area during their pediatric office visit. This assessment captures and reveals nonverbal communication gestures in clusters which help determine a person's state of mind or emotion by using multiple gestures, not just one. For example, crossing your arms at your chest can be a sign of being resistant and close-minded, but if the person's shoulders are raised and their teeth are chattering, they are cold and not resistant. Finally, congruence measures whether the spoken words match the tone and body language? After someone falls, and they say they are fine, are they grimacing or is their voice shaky?

### Yesterday's Paper and Pencil—Today's 21st Century Artificial Intelligence-Enabled Tech

This mental health assessment is superior to the traditional paper and pencil screener as demonstrated by the following facets:

1. An Age-Appropriate Game Platform that Collects Mental Health Data and Achieves Equally Consistent, Yet More Informative Data than Manually-Scored Mental Health Screeners:

A gamified platform for a mental health assessment of children and young adults allows for the collection of the largest data set for evaluation and result in similar or better indications of mental health concerns. The significance in creating a game is to relax the user, and foster unscripted responses. Relaxed users have comfort within the game platform but are not distracted from health assessment. This allows the collection of natural language data, facial expressions and voice data.

Working with a team of experts, several questions are generated that are aimed at family risk factors (disruptions), depression and anxiety signs and symptoms. Those are then sequenced and compared to the same scales within the Pediatric Symptom Checklist (PSC) (the paper and pencil equivalent). The application development team works with children and young adults to determine color and design elements that are the most age appropriate.

During the testing phase, children engage with the gamified assessment and complete the PSC. Both sets of data are analyzed to determine similarities and differences. Researchers evaluate quantity and scale of responses according to signs of family risk, depression and anxiety. Researchers also look for mild, moderate and severe levels, vs. the PSC which measure only major psychological impairment. In situations where the results are unclear or questionable, a personal review by a qualified psychologist takes place.

Throughout the testing phase, the psychometric engineers and research team continued to analyze patterns in the data. They looked for a percentage of symptoms that are mild, moderate and severe to measure and refine the game techniques to match, if not advance, the results of the PSC.

A team of psychologists separately watch greater than 25% of randomly selected videos and watch for scientifically mild and moderate symptoms to validate machine results. The human results are compared to the machine learning answers.

### 2. The Algorithm's Ability to Adequately Identify Emotion and Voice Shifts:

The computer can understand the age set of a 10-year-old up to a 14-year-old in natural language, emotion recognition and voice shifts. Children's speech recognition can be challenging mainly due to the inherent high variability in children's physical and articulatory characteristics and expressions. Variables can include psychological differences, developmental differences, and pronunciation differences. Speech patterns for children will differ from that of adults. Vocabulary of children are vastly different than that of adults.

A combination of supervised, unsupervised and reinforcement machine learning artificial intelligence with an initial list of words and phrases as a data set derived from existing science is used. This data set is used to train the machine to analyze collected speech from the game, 5-7 primary emotions and any major voice shifts. Working with a team of psychologists helps identify the 5-7 primary emotions that are mental health indicators. A team of psychologists separately watches greater than 25% of the videos randomly selected and match the emotion captured and voice shifts. The human results are compared to the machine learning answers. This team of psychologists measure machine reliability and identifies and assesses significant voice changes.

To accomplish this, a team of psychometric scientists are mapping the major markers noted for creating the assessment results. Speech to text transcripts are printed and a team of reviewers are looking for nonsensical words and phrases. If the content is legible and words and phrases are understandable, it is clear that the machine can understand the human and therefore begin flagging for keywords within the gamified interview.

3. A New Youth Designed Baseline that Develops a Revolutionary Psychometric Indicator and Reduces Bias from Current Assessment Measures:

One in five children suffers from a diagnosable mental health condition and less than 80% of those will receive support. It is expected to have a percentage consistent with current public health standards, yet this assessment provides the ability to more accurately understand the actual issue vs “behavior problems.”

To attain this, a team of developers review the raw data collected during the game including natural language, emotion recognition and voice shifts. The psychometric research team then classifies the data into known risk and protective factors with mild, moderate and severe levels. The research team then develops a scale of warning that takes into account frequency and decisiveness of the words. For example: I am sad vs. I want to die. A team of psychologists takes the data collected and compares it to the machine learning answers vs. paper-based answers.

### Vision and Impact

The vision surrounding this tool is to create a new path to knowledge of signs and symptoms that reduce bias, are broadly accessible and revolutionize how society views prevention and early intervention.

Suicide is the second leading cause of death among youth ages 10-24 and the statistics are worsening. While 90% of suicide fatalities within this group had an underlying mental illness, the average time from onset of a mental health condition to treatment is 10 years. This assessment dramatically shortens the time to treatment and significantly impacts treatment plans and options available for the child and their family.

This assessment results in a dramatically updated screening tool that is launched into pediatric offices, changing the landscape for mental health diagnoses and treatment among a vulnerable youth population. Being able to truly evaluate child functioning before it becomes a problem, improves the quality of families lives in the short term and for generations to come.

Several alternatives, modifications, variations, or improvements therein may be subsequently made by those skilled in the art, which are also intended to be encompassed by the following claims.

What is claimed is:

1. A computerized system for tracking a patient's mental health condition, the computerized system comprising:
a. an apparatus that displays queries and prompts of a computerized mental health assessment in a gamified manner, the displayed queries and prompts differing based on the age of the patient;
b. a user interface that provides the patient with a mechanism to provide answers and reactions to the queries and prompts;
c. an input device that enables a computer to collect a first set of data comprising the answers and reactions of the patient;
d. a central processing unit to retrieve and execute instructions, the central processing unit incorporating a computer assisted qualitative data analysis software, the data analysis software including artificial intelligence to analyze answers and reactions of the patient;
e. a memory including an electronic holding place for the raw data and instructions required for the central processing unit to perform data analysis;
f. an output device that enables the computer to transmit to a third party a second set of data, representing the patient's mental health condition, the second set of data being generated by the data analysis software and received from the central processing unit;
g. a first computerized device that utilizes the second set of data to generate guidance for the third party; and
h. a second computerized device to transmit a notification to the third party by means of electronic communication.

2. The system of claim 1, wherein the input device comprises sensors adapted to detect audio and/or visual answers and reactions of the patient.

3. The system of claim 2, wherein the sensors comprise a camera and microphone.

4. The system of claim 1, wherein the displayed queries and prompts are configured for patients comprising the age of four through ninety-nine years.

5. The system of claim 1, wherein the queries and prompts are configured to display images and avatars and ask expanded questions.

6. The system of claim 1, wherein the artificial intelligence located on at least one local server and/or cloud server flags keywords provided by the patient via text and/or audio and analyzes the answers and reactions to the queries and prompts using natural language processing, facial affect, and/or voice intonation.

7. The system of claim 6, wherein the artificial intelligence is configured to analyze emotions, including but not limited to surprise, anger, happiness, and sadness.

8. The system of claim 1, wherein the second set of data is configured to assist the third party in drafting a personalized mental health recommendation.

9. The system of claim 1, wherein the notification lacks substantive information on the patient's mental health condition and the third party is required to log into the system to get the substantive information.

10. The system of claim 1, wherein steps (a)-(c) are completed on a first computer and steps (d)-(e) are completed on a second computer.

11. A computer display implemented method of tracking a mental health condition of a patient comprising:
a. displaying gamified queries and prompts pertaining to the patient's mental health status using an assessment platform, the assessment platform comprising a display differing based on the age of the patient;
b. collecting answers and reactions to the queries and prompts of the patient;
c. using artificial intelligence to analyze the patient's answers and reactions to the queries and prompts;
d. calculating a risk-protective score;
e. computer-generated guidance for a third party; and
f. sending a notification to the third party by means of electronic communication.

12. The method of claim 11, wherein the age of the patient is one of four years through ninety-nine years.

13. The method of claim 11, wherein the queries and prompts are configured to ask expanded answers.

14. The method of claim 11, wherein at least a portion of the answers and reactions of the patient are collected using audio and/or visual data.

15. The method of claim 11, wherein artificial intelligence located on at least one local server and/or cloud server flags keywords provided by the patient via text and/or audio and analyzes the answers and reactions to the queries and prompts using natural language processing, facial affect, and/or voice intonation.

16. The method of claim 11, wherein the risk-protective score is a ratio of the overall risk score and protective score, represented by a number, roman numeral, letter, color, mathematical symbol, scientific symbol, or other indicia on a scale representing factors indicative of a patient's mild, moderate, or severe mental health condition.

17. The method of claim 11, wherein the risk-protective score is a measurement of environmental risk factors, including at least one of Adverse Childhood Experiences/trauma exposure (ACE), social determinants, relationships, functioning (in school; at home; with peers), parenting, parental mental health, family disruptions, environmental risks (fear of discrimination), and Positive Childhood Experiences (PCE) (strengths, gifts, talent).

18. The method of claim 11, wherein multiple mental health conditions are tracked, including anxiety, depression, Attention Deficit Hyperactivity Disorder (ADHD), suicidality, relationships, family disruptions, functioning in family, school and peers, Adverse Childhood Experiences (ACE), Positive Childhood Experiences (PCE), and positive findings.

19. The method of claim 11, further comprising using the computer-generated guidance to develop a personalized recommendation.

20. The method of creating a community health database comprising:
practicing the method of claim 11 as many times needed to create a quantified analysis of the community's mental health as one entity.
